(19) United States
(12) Patent Application Publication
(10) Pub. No.: US 2009/0209541 A1
(43) Pub. Date: Aug. 20, 2009

(54) APREPITANT COMPOSITIONS

(75) Inventors: Paras Jain, Hyderabad (IN); Umesh V. Barabde, Hyderabad (IN); Ravinder Kodipyaka, Hyderabad (IN); Indu Bhushan, Hyderabad (IN); Vijayavitthal Thippannachar Mathad, Hyderabad (IN); Pravinchandra Jayantilal Vankawala, Hyderabad (IN); Kolla Naveen Kumar, Hyderabad (IN); Arunagiri Muthulingam, Hyderabad (IN); Gangula Srinivas, Hyderabad (IN); Chlamala Subrahmanyeswara Rao, Hyderabad (IN); Elati Ravi Ram Chandrasekhar, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: Dr. Reddy's Laboratories Ltd., Hyderabad, AP (IN); Dr. Reddy's Laboratories, Inc., Bridgewater, NJ (US)

(21) Appl. No.: 12/305,133

(22) PCT Filed: Jun. 18, 2007
(86) PCT No.: PCT/US07/71476

§ 371 Date: Dec. 16, 2008

Related U.S. Application Data
(60) Provisional application No. 60/805,023, filed on Jun. 16, 2006.

(51) Int. Cl. A61K 31/5377

(52) U.S. Cl. 514/236.2

(57) ABSTRACT

Pharmaceutical compositions comprising aprepitant, wherein aprepitant solubility in aqueous media is enhanced.

### INTRODUCTION TO THE INVENTION

The present invention relates to powder compositions comprising aprepitant with improved solubility properties. More specifically, the invention relates to powder compositions of aprepitant with improved physicochemical characteristics, which help in the effective delivery of aprepitant. Methods for preparing the powder compositions are also described along with the methods of using these compositions for the treatment of a variety of conditions where aprepitant finds use, including emesis.

Aprepitant is a NK1 receptor antagonist chemically described as 5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one, and by the structural Formula I.

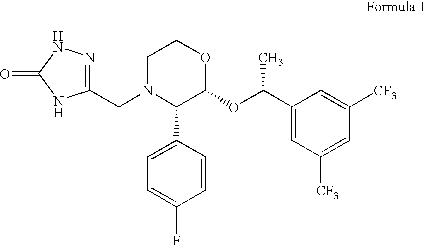

Aprepitant is a white to off-white crystalline solid, which is practically insoluble in water, sparingly soluble in ethanol and isopropyl acetate and slightly soluble in acetonitrile.

Aprepitant is approved internationally for the treatment of emesis associated with chemotherapy and is commercially available in the United States as EMEND® capsules from Merck, containing 40 mg, 80 mg and 125 mg of aprepitant for oral administration.

U.S. Pat. Nos. 6,096,742 and 6,583,142 describe two polymorphic forms of aprepitant, viz. Form I and Form II. Form I is said to be thermodynamically more stable than Form II, and has lower solubility as compared to Form II. Aprepitant is a molecule having poor solubility and poor permeability characteristics. Additionally, the delivery of aprepitant is also fraught with inter-patient variability when delivered as a tablet formulation, thereby requiring a nanoparticulate capsule-based composition to overcome this problem. The poor solubility of aprepitant in aqueous media and poor delivery characteristics pose a tremendous challenge to the pharmaceutical formulation scientist in its delivery in adequate concentrations into the systemic circulation.

International Application Publication No. WO 03/049718 addresses the issue of the poor delivery characteristics of aprepitant and discloses a nanoparticulate composition of aprepitant or a salt thereof, having adsorbed on its surface at least one surface stabilizer in an amount sufficient to maintain an effective average particle size of less than about 1000 nm. The commercially available composition of aprepitant, EMEND, could be based on this technology but suffers from a low bioavailability of about 60% in human beings.

The rate of dissolution of a poorly soluble drug is a rate-limiting factor in its absorption by the body. It is generally known that a reduction in the particle size of an active ingredient can result in an increase in the dissolution rate of such compounds through an increase in the surface area of the solid phase that comes in contact with the aqueous medium. Different active ingredients demonstrate an enhancement in dissolution rate to different extents. There is no way to predict the extent to which the dissolution rate of an active will be enhanced through particle size reduction or what is the desired particle size for achieving the desired bioavailability characteristics.

Other approaches to improve the solubility properties of active compounds include the use of emulsifiers, solubilizers, coprecipitates or solid dispersions, premixes, inclusion and other complexes, use of amorphous or alternate crystalline forms of the active and the like or combinations of these approaches.

The development of pharmaceutical compositions of aprepitant with improved solubility properties and improved bioavailability characteristics would be a significant improvement in the field of pharmaceutical science.

These and other unmet needs are addressed by the present invention.

### SUMMARY OF THE INVENTION

The present invention relates to powder compositions comprising aprepitant with improved solubility properties. More specifically, the invention relates to powder compositions of aprepitant with improved physicochemical characteristics, which help in the effective delivery of aprepitant.

An aspect of the present invention provides for powder compositions comprising aprepitant with improved solubility properties.

In an embodiment, the powder composition comprises a coprecipitate of aprepitant and a pharmaceutical carrier.

In another embodiment, the powder composition comprises an admixture of aprepitant and a surfactant.

In a further embodiment, the powder composition comprises aprepitant and a cyclodextrin.

In a further aspect, the powder composition comprises aprepitant with improved solubility properties, optionally with pharmaceutically acceptable excipient including emulsifiers, surfactants, wetting agents, crystallization inhibitors and the like, to provide improved wetting and solubility properties.

In a further embodiment, the powder composition of the present invention comprises aprepitant in amorphous or crystalline form, or the combinations thereof, optionally with pharmaceutically acceptable excipient.

Processes for preparing aprepitant with improved solubility properties, and compositions comprising the aprepitant with improved solubility properties are also described.

The powder compositions comprising aprepitant with improved solubility properties can be used for the treatment of a variety of medical conditions where aprepitant finds use.

An embodiment of the invention provides a pharmaceutical composition, comprising a solid premix comprising aprepitant and at least one pharmaceutical excipient and providing an aqueous solubility or dissolution rate of aprepitant that is greater than the aqueous solubility or dissolution rate of crystalline aprepitant.

Another embodiment of the invention provides a pharmaceutical composition comprising aprepitant particles having a mean particle size greater than about 2 μm and an aqueous solubility or dissolution rate of aprepitant that is greater than the aqueous solubility or dissolution rate of crystalline aprepitant.

In a further embodiment, the invention provides a pharmaceutical composition comprising aprepitant particles having a weight ratio of aprepitant crystalline Form I to aprepitant Form II about 5:95 to about 95:5.

### BRIEF DESCRIPTION OF THE DRAWINGS

FIG. 1 is an X-ray diffraction (“XRD”) pattern of amorphous aprepitant.

FIG. 2 is an X-ray diffraction pattern of a coprecipitate of aprepitant with polyethylene glycol in the weight ratio of 2:1.

FIG. 3 is an X-ray diffraction pattern of a coprecipitate of aprepitant with polyethylene glycol in the weight ratio of 3:1.

FIG. 4 is an X-ray diffraction pattern of a coprecipitate of aprepitant with polyethylene glycol in the weight ratio of 1:1.

FIG. 5 is an X-ray diffraction pattern of a coprecipitate of aprepitant with povidone in a ratio of 1:1, prepared using dichloromethane as solvent.

FIG. 6 is an X-ray diffraction pattern of a coprecipitate of aprepitant with povidone in a weight ratio of 1:1, prepared using a mixture of dichloromethane and methanol as a solvent.

FIG. 7 is an X-ray diffraction pattern of a coprecipitate of aprepitant with povidone in a weight ratio of 3:1, prepared using dichloromethane as a solvent.

### DETAILED DESCRIPTION OF THE INVENTION

The present invention relates to powder compositions comprising aprepitant with improved solubility properties.

Unless mentioned otherwise, all embodiments of the invention can be used for the delivery of aprepitant or any of its pharmaceutically acceptable salts, solvates, enantiomers or mixtures thereof, without limitation.

The present invention relates to powder compositions comprising aprepitant.

The term “powder compositions” as used herein refers to either a powder of aprepitant itself of defined physicochemical characteristics or a composition of aprepitant along with other excipients in the form of, for example, coprecipitates, premixes, solid dispersions, admixtures with surfactants and/or cyclodextrins, particles of a defined particle size along with, for example, emulsifiers and wetting agents, and the like.

“Aprepitant with improved solubility properties” in the context of the present invention refers to aprepitant or an aprepitant powder composition that has a higher solubility and/or dissolution rate, as compared to aprepitant in its crystalline form and having a comparable particle size distribution. Such improved solubility properties of aprepitant can be obtained by use of emulsifiers, solubilizers, coprecipitates or solid dispersions, premixes, inclusion and other complexes, use of amorphous or alternate crystalline forms, and the like, including combinations thereof.

The term “pharmaceutical composition” as used herein refers to formulations comprising aprepitant powder compositions as described above along with one or more additional pharmaceutically acceptable excipients required to convert the powder compositions of aprepitant into compositions for the delivery of aprepitant.

The term “solubility properties” as used herein refers to either an improvement in the solubility of aprepitant, or a modification in the rate of dissolution or a modified absorption of aprepitant.

The term “premix” as used herein refers to a powder composition of aprepitant in intimate or non-intimate mixture with one or more pharmaceutically acceptable excipients. In one aspect, the term premix is used for a powder composition of aprepitant wherein the aprepitant is uniformly distributed over a pharmacologically inactive carrier through an adsorption process.

In an embodiment, the powder composition comprises a coprecipitate of aprepitant and a pharmaceutical carrier.

The terms “coprecipitate” and “solid dispersions” as used in this invention are synonymous and are intended to mean a dispersion of aprepitant in an inert carrier or matrix in a solid state prepared by a melting (fusion), solvent or combined melt-solvent method.

Aprepitant may be prepared in different forms as particles of various sizes. According to one embodiment of the invention, aprepitant particles have a mean particle size greater than about 2 μm, or particle size greater than about 2 μm and less than about 500 μm. The term “particles” as used herein refers to individual particles of aprepitant or particles or aprepitant compositions. Thus, according to this embodiment aprepitant particles having a mean particle size greater than about 2 μm, or a mean particle size greater than about 2 μm and less than about 500 μm, are useful in providing improved solubility properties.

As used herein, the term “mean particle size” refers to the distribution of aprepitant particles wherein about 50 volume percent of all particles measured have a particle size less than the defined mean particle size value and about 50 volume percent of all measurable particles measured have a particle size greater than the defined mean particle size value; this can be identified by the term “D₅₀.” Similarly, a particle size distribution where 90 volume percent of the particles have sizes less than a specified size is referred to as “D₉₀” and a distribution where 10 volume percent of particles have sizes less than a specified size is referred to as “D₁₀.” The desired particle size range material is obtained directly from a synthesis process or any known particle size reduction processes can be used, such as but not limited to sifting, milling, micronization, fluid energy milling, ball milling, and the like.

Bulk density as used herein is defined as the ratio of apparent volume to mass of the material taken, called untapped bulk density, and also the ratio of tapped volume to mass of material taken, called tapped bulk density. A useful procedure for measuring these bulk densities is described in United States Pharmacopeia 24, Test 616 (Bulk Density and Tapped Density), United States Pharmaceopeial Convention, Inc., 2000.

Carr index as used herein is defined as the percent compressibility which is a percentage ratio of the difference between tapped bulk density and initial bulk density to tapped bulk density. Carr index values between 5-15% represent materials with excellent flowability, values between 18-21% represent fair-flowability and values above 40% represent very poor flowability.

Hausner ratio used herein is defined as the ratio of tapped to untapped bulk densities. A Hausner ratio less than about 1.2 indicates good flow properties, while a ratio greater than about 1.5 indicates poor flow properties.

In one aspect, the powder compositions of aprepitant of a defined particle size and distribution can be either of crystalline Forms I or II or can comprise a combination of crystalline Forms I and II. Further, aprepitant powder compositions with aprepitant in an amorphous form, alone or in combination with any other polymorphic form, with the above defined particle size and distribution are useful in the practice of this invention. Ratios of one form to another can range from about 100:0 to 0:100 w/w of any of the forms of aprepitant. In an embodiment, a weight ratio of Form I to Form II ranges between about 5:95 to about 95:5, or about 30:70 to about 70:30, or about 50:50. Processes for preparing various ratios of Form I to Form II are described in International Application No. PCT/US20071065474. For example, a composition containing a ratio of 50:50 by weight can be prepared by dissolving 40 g of aprepitant in 600 ml of acetone at about 60° C., cooling to about 1-5° C. and adding 600 ml of water. Alternatively, a composition having a 50:50 weight ratio can be prepared by completely distilling solvent at about 50° C. from a solution containing 5 g of aprepitant, 250 ml of dichloromethane, 5 ml of methanol and 0.5 ml of 50% aqueous ammonia.

An amorphous form of aprepitant is obtained from processes such as but not limited to dissolving aprepitant in a suitable solvent or solvent mixture and removing the solvent or solvent mixture in a controlled manner at controlled conditions such as temperature, pressure, and melting and flash cooling, solvents that are useful in the processes including methanol, ethanol, propanol, isopropanol, butanol, isobutanol, higher alcohols, benzene, toluene, acetone, chloroform, carbon tetrachloride, dichloromethane, and the like.

In another embodiment, the powder composition comprises an admixture of aprepitant and at least one surfactant. In yet another embodiment of the invention, improved solubility properties of aprepitant are provided by combining the aprepitant particles of a defined particle size and distribution in combination with a surfactant. The term “surfactant” is used synonymously with the terms “emulsifier”, “surface active agent”, “wetting agent” and the like and is intended to mean an excipient which, when in contact with the aprepitant particles, provides for their improved wettability. Weight ratios of aprepitant to emulsifier may range from about 1:50 to 50:1.

Without being bound by any particular theory, such materials are thought to adsorb superficially onto the particles of aprepitant, generally without any chemical reaction, and upon coming in contact with a fluid medium provide for rapid wetting of the active particle. The improved wetting in addition to a controlled particle size distribution provides enhanced solubility properties.

Such materials include but are not limited to anionic, cationic and nonionic surfactants. Anionic surfactants include materials such as carboxylates, acyl lactylates, ether carboxylates, sulphur- and phosphorus-containing anionic surfactants: sulphonates, phosphoric acid esters, chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate and sodium dodecyl sulfate (SLS or SDS). Cationic surfactants include materials such as quaternary ammonium salts, ethoxylated amines, cetylpyridinium chloride monohydrate and hexadecyl trimethylammonium bromide and the like. Nonionic surfactants include materials such as ethylene glycol esters, propylene glycol esters, glyceryl esters, polyglyceryl esters, sucrose esters, ethoxylated esters (PEGs), N-decanoyl-N-methylglucamine, octyl a-D-glucopyranoside, n-Dodecyl b-D-maltoside (DDM), polyoxyethylene sorbitan esters like polysorbate 10, 20, and 21, polyethyleneglycol-80 sorbitan laurate, polysorbate 80, 81, 40, 60, 61, 65, and 85, and the like. Amphoteric surfactants include materials such as acrylic acid derivatives, substituted alkylamines and phosphatides. Other surfactants include amine oxides, perflurinated alkyl derivatives, starch derived surfactants and polymeric surfactants.

Phospholipids which find use in the practice of the present invention as emulsifiers include lecithins or phosphatidyl cholines such as but not limited to dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC) and dilignoceroylphatidylcholine (DLPC), phosphatidyl ethanolamines like dioleoylphosphatidylethanolamine and di-stearoyl-phosphatidylethanolamine (DSPE), phosphatidylglycerols, phosphatidylserines, phosphatidylinositols and lysophosphatidyl derivatives. Combinations of one or more emulsifiers from the same class of compounds as well as from different classes are within the scope of this invention.

Compositions with surfactants can be prepared using techniques such as: simple admixture of aprepitant with surfactant; or grinding or milling aprepitant with surfactant; or dissolving surfactant in a solvent and adding to aprepitant and removing the solvent; or dissolving aprepitant and surfactant in a solvent or solvent mixture and removing solvent or solvent mixture slowly or by flash evaporation or freeze drying. Solvents that are useful in the process include methanol, ethanol, propanol, isopropanol, butanol, isobutanol, higher alcohols, benzene, toluene, acetone, chloroform, carbon tetrachloride and dichloromethane.

In another embodiment of the invention, powder compositions of aprepitant are provided comprising aprepitant along with at least one surface stabilizer. Surface stabilizers are substances that physically adhere to the surface of the compound, but do not chemically react with the drug itself. Individually adsorbed molecules of the surface stabilizer are essentially free of intermolecular cross-linkages. Surface stabilizers can be selected from known organic and inorganic pharmaceutical excipients, such materials include but are not limited to: gelatin, casein, lecithin (phosphatides), dextran; gums such as agrose, gum arabic, ghatti, karaya, tragacanth; acacia; cholesterol, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available “Tween™ products” such as Tween 20 and Tween 80 from ICI Speciality Chemicals); polyethylene glycols, polyoxyethylene stearates, colloidal silicon dioxide, phosphates, sodium dodecylsulfate; celluloses such as carboxymethylcellulose calcium, carboxymethylcellulose sodium, methylcellulose, hydroxyethylcellulose, hydroxypropylcellulose, hydroxypropylmethyl-cellulose, hydroxypropylmethyl-cellulose phthalate, noncrystalline cellulose; magnesium aluminium silicate, triethanolamine, polyvinyl alcohol (PVA), polyvinylpyrrolidone (PVP); N-methylglucamide; n-decylp-D-glucopyranoside; n-decyl P-D-maltopyranoside; n-dodecyl-D-glucopyranoside; n-dodecyl-D-maltoside; heptanoyl-N-methyl-glucamide; n-heptyl-p-D-glucopyranoside; n-heptyl-D-thioglucoside; n-hexyl P-D-glucopyranoside; nonanoyl-N-methylglucamide; n-noyl-D-glucopyranoside; octanoyl-N-methylglucamide; n-octyl-D-glucopyranoside; octylP-D-thioglucopyranoside; and the like. All of the classes of emulsifiers as described above can also be used as surface stabilizers without limitation.

The concentration of the one or more surface stabilizers can vary from about 0.01% to about 90%, or from about 1% to about 90%, by weight based on the total combined dry weight of the drug substance and surface stabilizer.

Various polymers, low molecular weight oligomers and natural products acting as surface stabilizers further include both hydrophilic and hydrophobic materials. The amounts and ratios of the emulsifiers or surface stabilizers required to provide the desired solubility properties of aprepitant can be decided based on the particular excipient and aprepitant combination.

With respect to the preparation of the powder compositions of the invention, the particle size can be reduced by any method for the reduction of particle sizes including but not limited to fluid energy mill or “micronizer,” ball mill, colloidal mill, roller mill, hammer mill and the like. The principal operations of conventional size reduction are milling of the feed stock material and sorting of the milled material by size. In an embodiment, feedstocks used for milling operations comprises but are not limited to crystals, powder aggregates, coarse powders of either crystalline or amorphous aprepitant, and the like. Aprepitant particles can be separated by particle size using various techniques such as cyclonic techniques, centrifugation techniques, and the like.

In one embodiment, a fluid energy mill or “micronizer” is found to be useful for its ability to produce particles of small size in a narrow size distribution. Micronizers use kinetic energy of collisions between particles suspended in a rapidly moving fluid (typically air) stream to cleave the particles.

In another embodiment, an air jet mill is found to be useful as an example of a fluid energy mill. The suspended particles are injected under pressure into a recirculating particle stream. Smaller particles are carried aloft inside the mill and swept into a vent connected to a particle size classifier such as a cyclone separator.

Particle sizes of aprepitant obtained from the above techniques are generally greater than about 2 μm. Other physicochemical characteristics of these powder compositions such as for example bulk density, flow properties, Carr index, Hausner ratio, aspect ratio, compressibility of these particles are in the ranges that aid processability and result in desired physicochemical properties of compositions and dosage forms like hardness, friability, solubility properties and bioavailability. These and other physicochemical properties of the powder compositions of aprepitant, either alone or in combination with any of the other embodiments described above are all within the scope of this invention without limitation. Any modifications required to be made to these physicochemical properties to improve further processing are also within the scope of this invention.

The surface stabilizers and emulsifiers as described above can be contacted with the compound before, during or after size reduction of the compound. Thus according to this embodiment, a slurry of the aprepitant particles of a defined particle size distribution can be prepared in a solution of the emulsifier or surface stabilizer. The particles thus coated with the emulsifier or stabilizers can be then recovered by various methods and dried or used for further processing such as that forming a pharmaceutical composition. Alternatively, particles of aprepitant of a defined particle size distribution can be granulated with a solution of the emulsifiers or stabilizers in appropriate solvents. The solvents used for the preparation of the solution of the emulsifiers or surface stabilizers can be aqueous, or an organic solvent or a mixture of solvents may be used.

In an embodiment, the powder composition comprises aprepitant and at least one cyclodextrin.

Cyclodextrins that may be used in the present invention include but are not limited to natural cyclodextrins and their derivatives, including the alkylated and hydroxyalkylated derivatives and the branched cyclodextrins. Examples of useful cyclodextrins are hydroxypropyl beta cyclodextrin, hydroxyethyl beta cyclodextrin, hydroxypropyl gamma cyclodextrin, hydroxyethyl gamma cyclodextrin, dihydroxypropyl beta cyclodextrin, glucosyl beta cyclodextrin, diglucosyl beta cyclodextrin, maltosyl beta cyclodextrin, maltosyl gamma cyclodextrin, maltotriosyl beta cyclodextrin, maltotriosyl gamma cyclodextrin and dimaltosyl beta cyclodextrin, and mixtures thereof such as maltosyl beta cyclodextrin/dimaltosyl beta cyclodextrin.

Coprecipitates, solid dispersions or inclusion complexes are prepared by co-melting aprepitant and one or more excipients and cooling; or dissolving aprepitant and one or more excipients in a solvent or solvent mixture and removing the solvent or solvent mixture slowly or by flash evaporation or under vacuum. Solvents that are useful in the process include water, methanol, ethanol, propanol, isopropanol, butanol, isobutanol, higher alcohols, benzene, toluene, acetone, chloroform, carbon tetrachloride, dichloromethane and combinations thereof.

In an embodiment, aprepitant is formulated as a “premix” which refers to a combination, such as a blend, or granules, of aprepitant with one or more pharmaceutically acceptable excipients.

An embodiment of an aprepitant premix of the invention is manufactured by dissolving aprepitant in a suitable solvent or solvent mixture such as water, isopropyl alcohol, acetic acid, acetone, anisole, ethanol, ethyl acetate, isopropyl acetate, dichloromethane and the like, including mixtures thereof, followed by the recovery of the material by any suitable means. The solvent mixture may contain an antioxidant. The alcoholic or hydroalcoholic or organic solvent mixture containing aprepitant is further adsorbed onto an excipient or a mixture of excipients using a suitable means, such as a rapid mixer granulator or planetary mixer or mass mixer or ribbon mixer or fluid bed processor or and the like. The aprepitant solution can be added to the mixture of excipients rapidly or gradually, as desired. The mode of addition can be simple pouring or more refined techniques such as pumping using a positive displacement pump or sprinkling or spraying onto the surface of the mixture of excipients. The solution of aprepitant in the solvent or solvent mixture can be added to the excipient or mixture of excipients either at the temperature of solubilization or at another temperature as desired. The wet mass thus produced is dried under controlled conditions to obtain an optimum loss on drying (LOD) using desired means such as a tray drier or fluid bed drier or rotary cone vacuum drier or agitated thin film drying equipment or lyophilization or the like. The drying temperature can frequently be made lower by applying a reduced pressure. The blend thus obtained herein referred to as an aprepitant premix may be further processed into various pharmaceutical dosage forms.

In another embodiment of the invention, a premix comprises a coprecipitate or solid dispersion of aprepitant and an excipient (or carrier). Solid dispersions can be made using hydrophilic or hydrophobic excipients or both. Commonly used excipients for solid dispersions include but are not limited to polyvinylpyrrolidone, polyethylene glycols, colloidal silicon dioxide, hydroxypropyl cellulose, hydroxypropylmethyl cellulose, sodium carboxymethyl cellulose, carboxymethyl cellulose, polyvinyl alcohol, dextran, lectins, carbopols, maltodextrins, lactose, fructose, polysaccharides, inositol, trehalose, maltose, raffinose, and lipids such as polyglycolized glycerides (Gelucire®, for example Gelucire 50/16, Gelucire 44/14) and their combinations in different ratios. Different molecular weight or viscosity grade variants of all of these are also within the scope of this invention. Other hydrophilic or hydrophobic materials acceptable for the preparation of solid dispersions are well within the scope of this invention without limitation as long as the materials affect the solubility properties of the aprepitant.

Processes for preparing coprecipitates comprising aprepitant and povidone are described in International Application Publication No. WO 2007/016582, which is incorporated herein in its entirety.

Several techniques useful for the preparation of solid dispersions or coprecipitates include solvent evaporation, melt-fusion, spray drying, spray freezing, spray congealing, melt extrusion, supercritical fluid precipitation and other techniques known in the art.

Solvents that are used in the preparation of solid dispersions using a solvent process comprising preparation of a solution comprising a carrier and solvent include but are not limited to alcohols such as methanol, ethanol, n-propanol, iso-propanol, n-butanol, iso-butanol and sec-butanol; acetone; dichloromethane; chloroform; and combinations thereof. Solvents used are either hydrous or anhydrous.

In yet another embodiment of premixes of the invention, microemulsions of aprepitant are provided wherein aprepitant, optionally together with one or more excipients, is incorporated in an oil such as mineral oil, vegetable oil, modified oils or combinations thereof and such compositions are combined with water or an aqueous media to form a microemulsion.

Particle size and size distribution, bulk density, flow properties, Carr index, Hausner ratio, aspect ratio, and compressibility of the particles of the above compositions are expected to be in the ranges that aid processability and result in desired physicochemical properties of dosage forms like hardness, friability, solubility properties and bioavailability.

Particle sizes of compositions of the present invention are greater than about 2 μm, or greater than about 2 μm and less than about 500 μm, and are useful in providing improved aprepitant solubility properties. A Carr index of particles of compositions of the present invention may be less than 40%, or less than 40% and more than 5%.

The powder compositions of this invention as described in the different embodiments above are useful in the preparation of pharmaceutical compositions for the delivery of aprepitant. As used herein, pharmaceutical composition means a composition for use in treating a mammal that includes aprepitant of defined particle size and is prepared in a manner that is appropriate for administration to a mammal, such as a human. A pharmaceutical composition contains one or more pharmaceutically acceptable excipients that are non-toxic to the mammal intended to be treated when the composition is administered in an amount effective to treat the mammal.

Aprepitant prepared according to any of the embodiments of the powder compositions above can be incorporated into pharmaceutical compositions or a combination of materials made by different embodiments can be used.

The pharmaceutical compositions may be in the form of encapsulated free flowing powders or granules; compressed solid dosage forms such as tablets, including chewable or dispersible or mouth dissolving, as well as the customary forms that are swallowed whole; pellets (extruded or fluidized) or beads or spheres or cores (water-soluble or insoluble or both) filled into sachets or capsules; enteric solutions, syrups, suspensions or dispersions; emulsions like micro-emulsions or multiple-emulsions; elixirs, troches, lozenges, lyophilized powders and the like.

Also the lyophilized powders or enteric solutions or suspensions or dispersions, emulsions like micro-emulsions or multiple-emulsions, of aprepitant can further be filled into hard or soft gelatin capsules.

The pharmaceutical compositions of the present invention may contain one or more diluents to increase the final composition mass so that it becomes easier for the patient and caregiver to handle.

Common diluents that can be used in pharmaceutical formulations comprise microcrystalline cellulose (MCC), silicified MCC (e.g. Prosolv™ HD 90), micro fine cellulose, lactose, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like.

The pharmaceutical compositions may further include a disintegrant. Disintegrants include but are not limited to methyl cellulose, microcrystalline cellulose, carboxymethyl cellulose calcium, carboxymethyl cellulose sodium (e.g. Ac-Di-Sol®, Primellose5), crospovidone (e.g. Kollidon®, Polyplasdone®), povidone K-30, guar gum, magnesium aluminum silicate, colloidal silicon dioxide (Aerosil®), polacrilin potassium, starch, pregelatinized starch, sodium starch glycolate (e.g. Explotab®) and sodium alginate.

Pharmaceutical compositions may further include ingredients such as but not limited to pharmaceutically acceptable glidants, lubricants, opacifiers, colorants, sweeteners, thickeners and other commonly used excipients.

In an embodiment, the pharmaceutical compositions of the present invention are manufactured as described below. The granules of actives are prepared by sifting the actives and excipients through the desired mesh size sieve and then are mixed, such as by using a rapid mixer granulator or planetary mixer or mass mixer or ribbon mixer or fluid bed processor or any other suitable device. The blend can be granulated, such as by adding a solution of a binder whether alcoholic or hydro-alcoholic or aqueous in a low or high shear mixer, fluidized bed granulator and the like or by dry granulation. The granulate can be dried using a tray drier or fluid bed drier or rotary cone vacuum drier and the like. The sizing of the granules can be done using an oscillating granulator or comminuting mill or any other conventional equipment equipped with a suitable screen. Alternatively, granules can be prepared by extrusion and spheronization or roller compaction. The dried granulate particles are sieved, and then mixed with lubricants and disintegrants and compressed into a tablets.

Alternatively the manufacture of granules of actives can be made by mixing the directly compressible excipients or by roller compaction. The blend so obtained is compressed using a suitable device, such as a multi-station rotary machine to form slugs, which are passed through a multimill or may be produced by methods of particle size reduction including but not limited to fluid energy mill or ball mill or colloidal mill or roller mill or hammer mill and the like, equipped with a suitable screen. The milled slugs of actives are then lubricated and compressed into tablets.

The powder compositions comprising aprepitant with improved solubility properties can be used for the treatment of a variety of medical conditions where aprepitant finds use, for example, in the treatment of emesis induced by radiation including radiation therapy such as in the treatment of cancer, and post-operative nausea and vomiting.

In yet another embodiment, the pharmaceutical compositions of the present invention may be formulated optionally with one or more therapeutic agent(s) for the treatment and relief of emesis or post-operative nausea and vomiting such as a 5-HT3 antagonist like ondansetron or granisetron or tropisetron, a dopamine antagonist such as metoclopramide or domperidone, or a GABA-B receptor agonist such as baclofen and the like.

The following examples will further illustrate certain specific aspects and embodiments of the invention in greater detail and are not intended to limit the scope of the invention in any manner. X-ray diffraction patterns described herein were obtained using copper K-α radiation (1.541 Å), and the patterns of the drawing figures have a vertical axis of intensity units and a horizontal axis that is the 2θ angle, in degrees.

### Example 1

### Preparation of Amorphous Aprepitant

35 g of aprepitant was dissolved in 300 ml of tetrahydrofuran to get a clear solution. This solution was spray dried using a spray drier (Jay Instruments & Systems Pvt Ltd., India, Model LSD-348-PLC) maintaining the feed rate at 110 ml per hour, aspiration rate at >1600 RPM to maintain negative pressure of 110-130 mm W.C., inlet temperature at 140° C., outlet temperature at 80° C. and atomization air pressure at 2.2 kg/cm². 20 g of dried substance was collected.

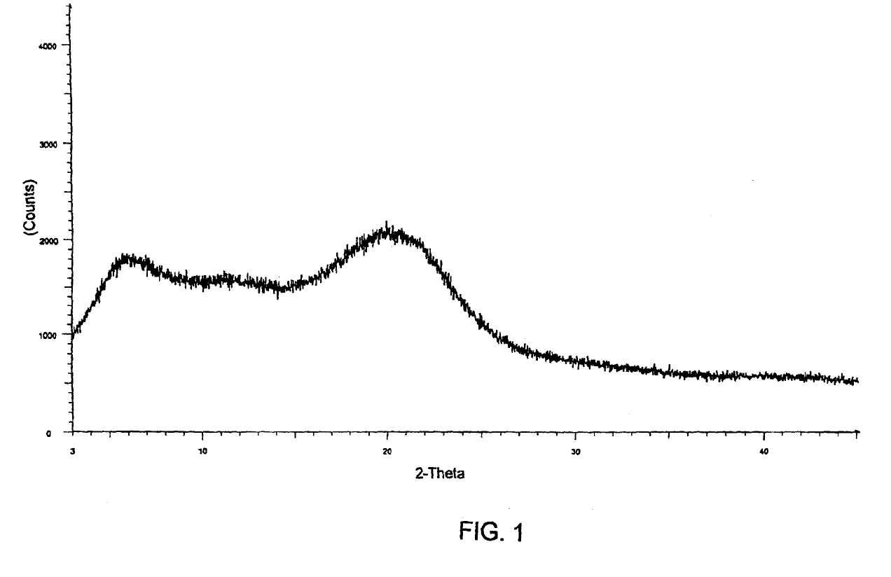

The XRD pattern of the sample demonstrates the amorphous nature as shown in FIG. 1.

### Example 2

### Coprecipitates of Aprepitant with Polyethylene Glycol

2 g of aprepitant and polyethylene glycol 6000 (1 g, 0.67 g and 2 g respectively) in different ratios of (2:1, 3:1 and 1:1 w/w) along with dichloromethane (300 ml, 320 ml and 190 ml respectively) were charged into separate round bottom flasks and stirred at a temperature 35-40° C. The mixtures were heated to reflux to obtain clear solutions. The solutions were filtered while hot using a Büchner funnel. The filtrates were transferred into three different Buchi Rotavapor flasks and the solvents were evaporated under vacuum at 45-50° C. to obtain coprecipitates of aprepitant with polyethylene glycol.

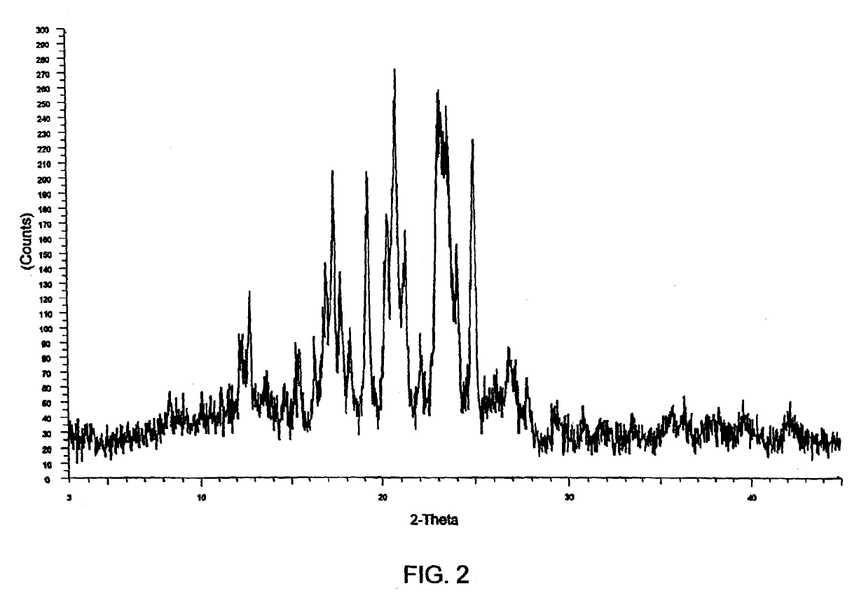

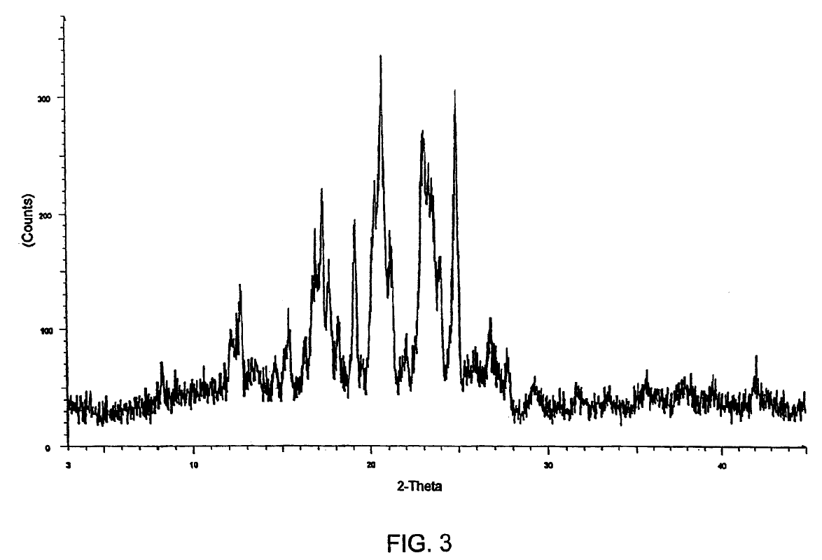

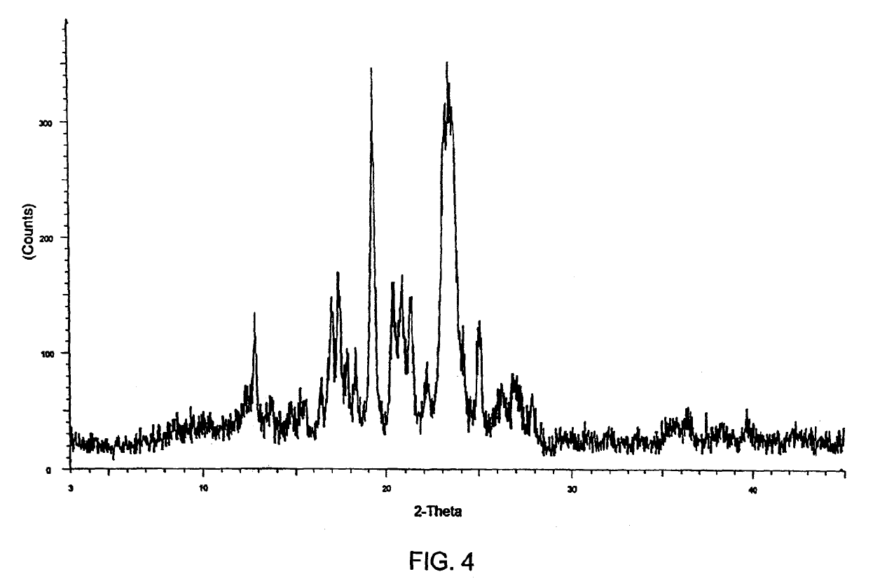

FIGS. 2 through 4 show the respective XRD patterns of these coprecipitates.

### Example 3

### Coprecipitate of Aprepitant with Povidone in a Ratio of 1:1 w/w Using Dichloromethane as the Solvent

1 g of aprepitant and 1 g of povidone (PVP K30) were dissolved in 200 ml of dichloromethane with the aid of heating to a temperature of 40° C. The solution was filtered in the hot condition and the dichloromethane was removed using distillation in a Buchi Rotovapor apparatus under a vacuum of 0-20 torr. 1.8 g of a dried coprecipitate of aprepitant with povidone was obtained.

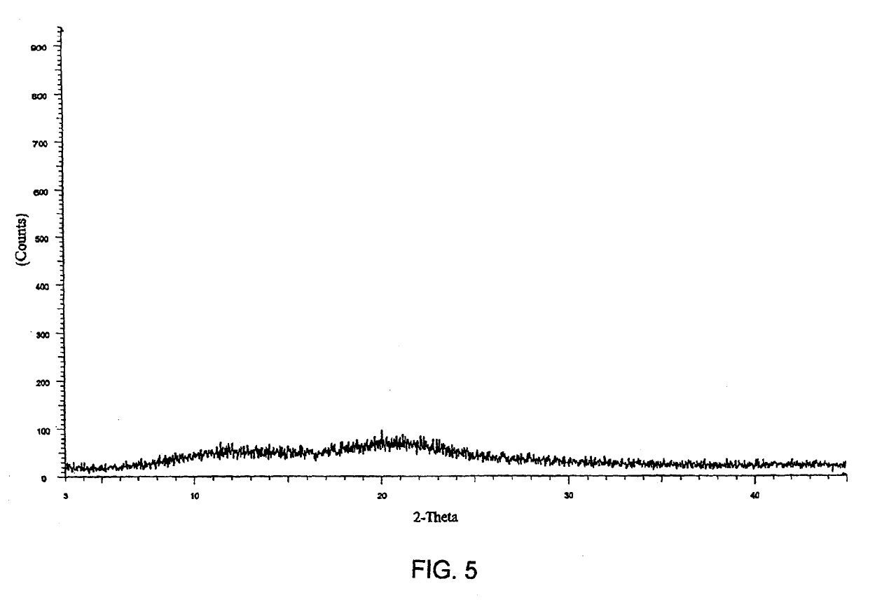

The XRD pattern of the sample demonstrates the amorphous nature of the coprecipitate, as shown in FIG. 5.

### Example 4

### Coprecipitate of Aprepitant with Povidone in a Ratio of 1:1 w/w Using a Combination of Dichloromethane and Methanol as the Solvent

5 g each of aprepitant and povidone (PVP K 30) were dispersed in 1000 ml of dichloromethane and stirred for 30 minutes. 20 ml of methanol was added and stirred for 5 minutes to get a clear solution. The solution was filtered through a flux calcined diatomaceous earth (Hyflow) bed and the solvents were removed using distillation in a Buchi Rotovapor apparatus under a vacuum of 0-22 torr. 10 g of a dried coprecipitate of aprepitant with povidone were obtained.

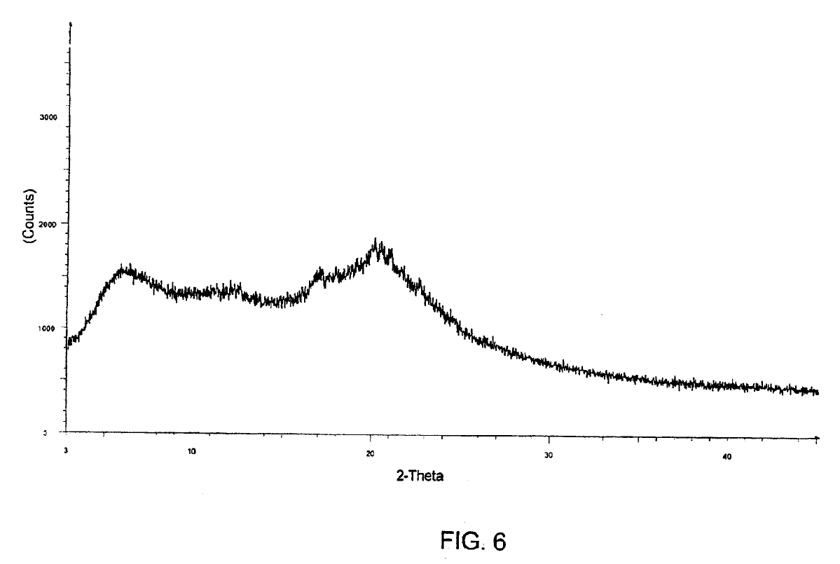

The XRD pattern of the sample demonstrates the amorphous nature of the coprecipitate, as shown in FIG. 6.

### Example 5

### Coprecipitate of Aprepitant with Povidone in a Ratio of 3:1 w/w Using Dichloromethane as the Solvent

1.5 g of aprepitant and 0.5 g of povidone (PVP K 30) were dissolved in 300 ml of dichloromethane with the aid of heating to a temperature of 40° C. The solution was filtered and the dichloromethane was removed using distillation in a Buchi Rotavapor apparatus under a vacuum of 2-20 torr. 1.8 g of a dried coprecipitate of aprepitant with povidone was obtained.

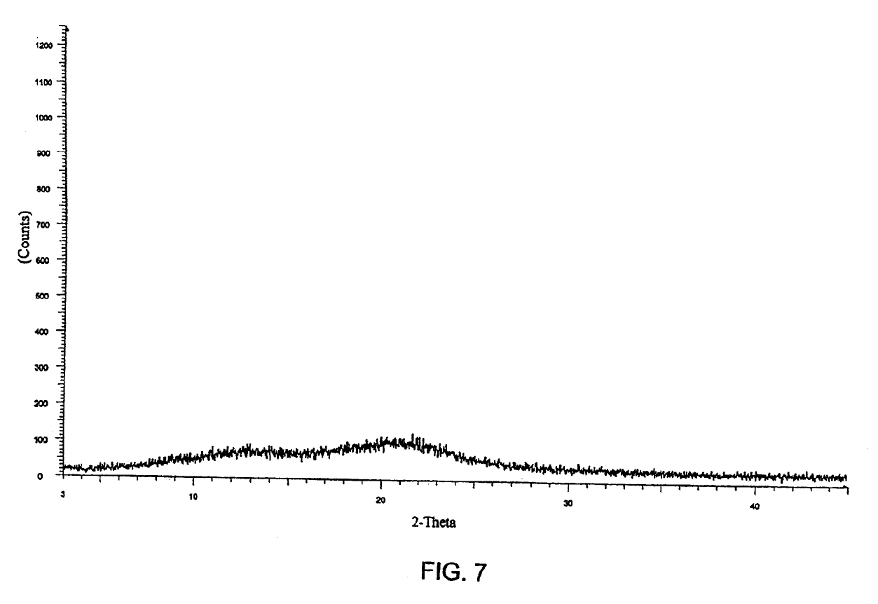

The XRD pattern of the sample demonstrates the amorphous nature of the coprecipitate, as shown in FIG. 7.

### Example 6

### Coprecipitate of Aprepitant with Povidone in a Ratio of 7:3 w/w

3.5 g of aprepitant and 1.5 g of povidone were taken into a round bottom flask and 140 ml of dichloromethane were added. The mass was stirred for 25 minutes at 27° C. 3.5 ml of methanol were added and stirring was continued for another 20 minutes. The mass was filtered through a celite bed and the filtrate distilled in a Buchi Rotavapor at a temperature of 45° C. and pressure of 300 mm Hg to yield 4.85 g of the coprecipitate.

### Example 7

### Powder Compositions of Aprepitant with Cyclodextrin (1:1 Molar Ratio)

2.12 g of β-cyclodextrin was dissolved in 100 ml of a 2:3 by volume ratio of water and methanol and 1 g of aprepitant was added and dissolved. The solution was shaken for 6 hours at 50° C. The solid was separated by filtration and dried in a tray drier at 50° C., until the loss on drying was 7.6% when measured at 80° C. using an infrared moisture balance.

### Example 8

### Powder Composition of Aprepitant with a Combination of a Cyclodextrin and Wetting Agent (1:1.5 Molar Ratio)

0.925 g of β-cyclodextrin was dissolved in 50 ml of water, 0.02 g of Poloxamer 407 (block copolymer of ethylene oxide and propylene oxide) was added and dissolved, 0.29 g of aprepitant was added to this solution and the mixture was kept on a shaking machine for 6 hours at room temperature. The solid phase was separated by filtration and dried in a tray drier at 50° C.

### Example 9

### Powder Composition of Aprepitant Containing a Polymeric Wetting Agent

500 mg of gelatin was dissolved in 50 ml of water and 100 mg of Poloxamer 407 was added and dissolved. 1 gram of aprepitant was granulated using the solution. Granules obtained were dried at 50° C.

### Example 10

### Capsule Composition of the Coprecipitate of Aprepitant with Povidone

A coprecipitate of aprepitant with povidone prepared according to Example 4, equivalent to 80 g aprepitant, is sifted through a 40 mesh ASTM sieve and is mixed with presifted 80 g of sucrose, 120 g of microcrystalline cellulose and 10 g of sodium starch glycolate, then blended with 5 g of magnesium stearate and 5 g of talc. An amount of the blend equivalent to 125 mg of aprepitant is filled into a hard gelatin capsule.

### Example 11

### Capsule Composition of the Coprecipitate of Aprepitant with PEG

A coprecipitate of aprepitant with PEG prepared according to Example 2 (1:1 w/w ratio), equivalent to 80 g of aprepitant, is sifted through a 40 mesh ASTM sieve and is mixed with pre-sifted 80 g of sucrose, 120 g of microcrystalline cellulose and 10 g of sodium starch glycolate, then blended with 5 g of magnesium stearate and 5 g of talc. An amount of the blend equivalent to 125 mg of aprepitant is filled into a hard gelatin capsule.

### Example 12

### Solubility of Powder Compositions of Aprepitant at 25° C. in Water

| | | Solubility |
|---|---|---|
| Identifier | Composition | (mg/ml) |
| A | Aprepitant (crystalline Form I) | 0.0005 |
| B | Aprepitant:PVP K30 (1:1 w/w) solid dispersion | 0.001 |
| C | Premix of Aprepitant and Aprepitant:PVP K30 | 0.001 |
| | (1:1 w/w) solid dispersion (1:6 w/w) | |
| D | Premix of Aprepitant and Aprepitant:PVP K30 | 0.001 |
| | (1:1 w/w) solid dispersion (1:2 w/w) | |
| E | Premix of Aprepitant and Aprepitant:PVP K30 | 0.001 |
| | (1:1 w/w) solid dispersion (3:2 w/w) | |
| F | Aprepitant:Polysorbate 80 (1:1 w/w) | 0.066 |
| G | Aprepitant:Gelucire 50/16 (1:1 w/w) | 0.063 |
| H | Aprepitant:beta-Cyclodextrin (1:1 molar ratio) | 0.001 |

### Manufacturing Process:

A. Aprepitant: Neat aprepitant was passed through a BSS # 80 mesh sieve, and its solubility in water was determined using a HPLC method.
B. Aprepitant: PVP K30 (1:1 w/w) solid dispersion: 2 g aprepitant and 2 g PVP K 30 were dissolved in a mixture of 200 ml dichloromethane and 20 ml methanol. This solution was dried in a rotary flash evaporator at 50° C. The dried solid dispersion was passed through a BSS # 80 mesh sieve.
C. Premix of A and B (1:6 w/w): Neat aprepitant and solid dispersion from B was mixed in the weight proportions of 1:6. This mixture was passed through a BSS # 80 mesh sieve.
D. Premix of A and B (1:2 w/w): Neat aprepitant and solid dispersion from B was mixed in the weight proportions of 1:2. This mixture was passed through a BSS # 80 mesh sieve.
E. Premix of A and B (3:2 w/w): Neat aprepitant and solid dispersion from B was mixed in the weight proportions of 3:2. This mixture was passed through a BSS # 80 mesh sieve.
F. Aprepitant: Polysorbate 80 (1:1 w/w): 1 g aprepitant and 1 g polysorbate 80 were mixed together by trituration. This mixture was passed through a BSS # 80 mesh sieve.
G. Aprepitant:Gelucire 50/16 (1:1 w/w): 1 g Gelucire 50/16 was melted by heating to 60° C. and 1 g aprepitant was added and mixed. This mixture was cooled to room temperature and the resultant mass was passed through a BSS # 80 mesh sieve.
H. Aprepitant:beta-Cyclodextrin (1:1 molar ratio): 2 g aprepitant and 4.25 g beta-cyclodextrin were dispersed in a mixture of 120 ml water and 120 ml methanol. This dispersion was dried in rotary flash evaporator at 60° C. The dried mass was passed through a BSS # 80 mesh sieve.
   Analytical method for determination of solubility of aprepitant.
   Chromatographic conditions: A liquid chromatograph equipped with variable wavelength detector and integrator.
Column: Hypersil® BDS C-8, 150×4.6×5 or equivalent.
Wavelength: 210 nm
Flow rate: 1.0 ml/minute
Column temperature: Ambient
Load: 20 μl
Diluent: Mixture of Acetonitrile and water in the ratio of 1:1.
Run time: 15 minutes.
Buffer: Dissolved 1.96 g ortho-phosphoric acid and 0.34 g of tetrabutylammonium hydrogen sulphate in 1000 ml water.
Mobile phase A: Buffer:Acetonitrile (80:20 v/v)
Mobile phase B: Acetonitrile:Buffer (80:20 v/v)

Gradient Program:

| Time | % A | % B |
|---|---|---|
| 0.0 | 55 | 45 |
| 15 | 55 | 45 |

Preparation of Standard Solution:

Weighed accurately 10.0 mg of aprepitant working standard into a 100 ml volumetric flask, dissolved and diluted to the volume with diluent (0.1 mg/ml final concentration).

### Sample Solution Preparation:

About 100 mg of aprepitant-containing sample was placed into a 100 ml volumetric flask and added 100 ml of desired pH buffer solution as prepared above, and cyclo-mixed the solution for about 15 minutes, filtered and filtrate solution was injected.

### Procedure:

Injected blank once, standard solution, all sample preparations in different pH solutions each twice into the chromatographic system. Aprepitant solubility in mg/ml was calculated using following formula:

Solubility in mg/mL=0.1(Average area of aprepitant peak from sample preparations in different pH)÷(Average area of aprepitant peak from standard solution)

### Example 13

### Capsule Formulations Comprising Aprepitant with Improved Solubility Properties

| Ingredient | | | | | | | | |
|---|---|---|---|---|---|---|---|---|
| from | | | | | | | | |
| Example | Quantity (mg/Capsule) | | | | | | | |
| 12 | F001 | F002 | F003 | F004 | F005 | F006 | F007 | F008 |
| A | 125 | — | 93.75 | 62.5 | 31.25 | — | — | — |
| B | 0 | 250 | 62.5 | 125 | 187.5 | — | — | — |
| F | — | — | — | — | — | — | — | 160 |
| G | — | — | — | — | — | 250 | — | — |
| H | — | — | — | — | — | — | 250 | — |
| MCC* | — | — | — | — | — | — | — | 220 |
| Lactose | 150 | 100 | 160 | 125 | 125 | 115 | — | — |
| Total | 275 | 350 | 316.25 | 312.5 | 343.75 | 365 | 250 | 380 |

*Microcrystalline cellulose

### Manufacturing Process:

Respective ingredients were mixed together and filled into size “0el’ capsules.

### In Vitro Dissolution Testing Data of Capsule Products

Medium: Purified water containing 2.2% SLS

Apparatus: USP type II (Paddle) from Test 711, “Dissolution,” in United States Pharmacopeia 29, United States Pharmacopeial Convention, Inc., Rockville, Md. (2006).

Volume: 900 ml

Rotation speed: 100 rpm

| Time | Cumulative % Drug Released | | | | | | | |
|---|---|---|---|---|---|---|---|---|
| (min.) | F001 | F002 | F003 | F004 | F005 | F006 | F007 | F008 |
| 0 | 0 | 0 | 0 | 0 | 0 | 0 | 0 | 0 |
| 15 | 48 | 15 | 64 | — | — | 5 | 11 | 54 |
| 45 | 70 | 32 | 84 | 54 | 69 | 42 | 67 | 78 |

1. A pharmaceutical composition, comprising a solid premix comprising aprepitant and at least one pharmaceutical excipient and providing an aqueous solubility or dissolution rate of aprepitant that is greater than the aqueous solubility or dissolution rate of crystalline aprepitant.

2. The pharmaceutical composition of claim 1, wherein a premix comprises a solid dispersion comprising amorphous aprepitant and at least one pharmaceutical excipient.

3. The pharmaceutical composition of claim 1, wherein a premix comprises a coprecipitate comprising aprepitant and at least one pharmaceutical excipient.

4. The pharmaceutical composition of claim 1, wherein a premix comprises a coprecipitate comprising aprepitant and a povidone.

5. The pharmaceutical composition of claim 1, wherein a premix comprises a coprecipitate comprising aprepitant and a povidone in a weight ratio about 1:4 to about 4:1.

6. The pharmaceutical composition of claim 1, wherein a premix comprises a coprecipitate comprising aprepitant and a povidone in a weight ratio about 1:1.

7. The pharmaceutical composition of claim 1, wherein a premix comprises a coprecipitate comprising aprepitant and a povidone in a weight ratio about 3:7.

8. A pharmaceutical composition comprising aprepitant particles having a mean particle size greater than about 2 μm and an aqueous solubility or dissolution rate of aprepitant that is greater than the aqueous solubility or dissolution rate of crystalline aprepitant.

9. The pharmaceutical composition of claim 8, wherein aprepitant particles comprise a mixture of aprepitant crystalline Form I and aprepitant crystalline Form II.

10. The pharmaceutical composition of claim 8, wherein aprepitant particles comprise amorphous aprepitant.

11. The pharmaceutical composition claim 8, wherein aprepitant particles comprise an admixture of aprepitant and a surfactant.

12. The pharmaceutical composition of claim 8, wherein aprepitant particles comprise an admixture of aprepitant and a surface stabilizer.

13. The pharmaceutical composition of claim 8, wherein aprepitant particles comprise an admixture of aprepitant and a cyclodextrin.

14. A pharmaceutical composition comprising aprepitant particles having a weight ratio of aprepitant crystalline Form I to aprepitant Form II about 5:95 to about 95:5.

15. The pharmaceutical composition of claim 14, wherein aprepitant particles have a weight ratio of aprepitant crystalline Form I to aprepitant Form II about 1:1.
